(19) United States
(12) Patent Application Publication
(10) Pub. No.: US 2021/0363600 A1
(43) Pub. Date: Nov. 25, 2021

(54) PRIMER GROUPS FOR DETECTING HYBRID RICE BACKBONE PARENT AND APPLICATION THEREOF

(71) Applicant: Institute of Food Crops, Hubei Academy of Agricultural Sciences, Wuhan (CN)

(72) Inventors: Haiya CAI, Wuhan (CN); Chunhai Jiao, Wuhan (CN); Shuo Zhang, Wuhan (CN); Gang Liu, Wuhan (CN); Haitao Jia, Wuhan (CN); Yuxia Lu, Wuhan (CN); Aiqing You, Wuhan (CN); Lei Zhou, Wuhan (CN); Li Zhou, Wuhan (CN)

(73) Assignee: Institute of Food Crops, Hubei Academy of Agricultural Sciences

(21) Appl. No.: 17/323,971

(22) Filed: May 18, 2021

(30) Foreign Application Priority Data
May 19, 2020 (CN) 202010427252.8

(51) Int. Cl. C12Q 1/6895

(52) U.S. Cl. CPC C12Q 1/6895; C12Q 2600/156; C12Q 2600/13

(57) ABSTRACT

Primer groups for detecting a hybrid rice backbone parent and application thereof. The primer groups according to the invention can distinguish any one of 29 backbone parent materials simply by judging based on a band pattern, without sequencing. Meanwhile, primers according to the invention have high polymorphism and can be applied to the purity detection of hybrid rice seeds for production, where only DNA is extracted from a sample indoor and then detected by using a molecular marker. Therefore, time, labor and cost are saved.

### CROSS-REFERENCE TO RELATED APPLICATIONS

The subject application claims priority on Chinese Application No. CN202010427252.8 filed on May 19, 202 in China. The contents and subject matter of the Chinese priority application is incorporated herein by reference.

### TECHNICAL FIELD

The invention belongs to the field of molecular genetics, and in particular, relates to primer groups for detecting a hybrid rice backbone parent and application thereof in the development of a rice identification code. The primer groups can be widely applied to fields such as purity identification and variety authenticity identification of hybrid rice seeds.

### BACKGROUND OF TECHNOLOGY

As one of the most important grain crops, rice feeds more than half of the population of the world. In China, about 70% of the population take rice as their staple food. Therefore, great attention has been always paid to the genetic improvement of rice varieties. In recent years, rice varieties newly examined and approved in China have been increasing year-by-year. For example, there were 565 rice varieties examined and approved nationwide throughout 2008, and 943 by 2018, where indica type rice accounted for 77.3% and hybrid rice accounted for 71.8%. A sharp increase in the number of new varieties has placed a greater demand on the variety identification and diversity analysis.

Before a rice variety is examined and approved, a DUS test needs to be conducted to find out its distinctive features from other existing varieties. A traditional DUS (distinctness, uniformity and stability) test for a new rice variety mainly depends on the identification of phenotypic parameters in the field. The DUS test has some defects, for example, the rice needs to be observed and recorded in the whole growth period, which is time-consuming and appears somewhat experiential; and an interaction between a genotype and an environment also leads to an unstable phenotype. In addition, with the acceleration of a breeding process, crop varieties have significantly increased. In particular, a minority of excellent backbone parents have been used repeatedly during modern breeding in recent years, leading to high genetic similarity among bred varieties. As a result, it is difficult to distinguish these varieties only by the phenotype, and the phenotype-based variety identification has been far from meeting the needs.

Also, an increase in the number of varieties in the market at present leads to problems in the intellectual property protection of varieties. Due to the repeated use of the minority of excellent parents during the modern breeding, intervarietal genetic background has become narrower, and a difference in phenotypic character has become smaller. For some varieties with a large market demand, some similar fake varieties would be sold in the market, leading to intellectual property disputes in sales; and due to high genetic similarity, it is difficult to distinguish these varieties by the phenotype. Therefore, there is also an urgent need of a new technique for solving the current problems in the aspect of variety property protection.

It is important to monitor the purity during the production and sales of hybrid rice varieties, since the mixing of seeds may induce losses in the quality and yield of rice, causing disputes between growers and marketing companies. At present, two-line hybrid rice accounts for about a half of the planting area of hybrid rice. Since the fertility transformation of a two-line sterile line depends on the natural environment (temperature and illumination), the fertility of the sterile line would become unstable if the temperature falls at a booting stage during the seed production of a two-line variety; and furthermore, the genetic shift of the sterile line during its propagation may also lead to unstable fertility, leading to self-fruitfulness of the sterile line and resulting in the mixing and purity reduction of hybrid seeds. During traditional purity detection, a growth-out test is used to determine the purity, which is then identified by phenotypic investigation after field planting. This method requires much time and effort, increasing the operating cost of seed enterprises.

Molecular marker is a biological technology developed in recent years and has been widely applied to fields such as crop genetics. A marker designed based on an indel sequence of a rice genome (hereinafter referred to as an ID marker) has advantages such as wide spreading, co-dominance, good repeatability, high polymorphism, stable amplification results, simple and practicable detection means; and furthermore, compared with the latest technologies such as array chip, the molecular marker has the advantage of low technical cost and can be conducted in ordinary small laboratories.

Therefore, a new approach to a series of problems faced in the rice production as mentioned above can be developed by analyzing the genetic diversity of the existing hybrid rice backbone parent using the ID marker and distinguishing a rice variety currently used in production at a genomic level.

### DESCRIPTION OF THE INVENTION

In the invention, indel sites are obtained by performing amplification and sequencing as well as comparison on sequences of some hybrid rice backbone parents, and the primers are designed on both sides of the indel sites to obtain ID markers, thereby establishing a set of primer groups capable of distinguishing some hybrid rice backbone parents that have been widely applied to the production at present, where the primer groups completely distinguish 29 parents based on the differences in the size of amplification bands. A result can be determined without performing sequencing on a target band and even without using the marker, thereby greatly reducing the workload of detection staff and the detection cost. The applicant made further utilization of the primer groups to establish identification codes for these materials, and meanwhile, explored their applications to the purity detection for the hybrid rice, thereby providing assistance to the production of hybrid rice.

An object of the invention is to provide primer groups capable of distinguishing a hybrid rice backbone parent. The primer groups consist of 9 pairs of primers, which are shown in Table 1.

Another object of the invention is to provide applications of the primer groups shown in Table 1. The primers can identify any of 29 backbone parent materials, or one or some of the primers can be selected to identify the purity of a combination of hybrid rice, or to establish an identification code for a rice variety.

To achieve the objects described above, the invention utilizes the following technical measures.

The applicant selected hybrid rice parental materials that had been widely used in the rice production at present, including a two-line sterile line, a three-line sterile line and a restorer line, and a total of 29 materials were used as study objects for developing their detecting primer groups. First, sequencing primers were designed randomly in the whole genome by using Nipponbare, a variety that had been sequenced; according to a pedigree relationship, two representative varieties having a distant genetic relationship, namely, a hybrid rice female parent Pei'ai 64S and a hybrid rice male parent Huazhan, with numerous combinations and wide promotion scope among the hybrid rice backbone parents were selected; the two varieties were subjected to sequence amplification and sequenced comparison by using the designed sequencing primers to obtain indel sites; and primers were designed by using the information of sequences on both sides of the sites to obtain ID markers. The markers were selected based on the principle of uniform distribution in a rice genome. The applicant performed PCR amplification on 29 materials and finally obtained 9 pairs of primers through final screening to constitute primer groups, each individually identified as having SEQ ID NO: 1 to SEQ ID NO:18, which are specifically as follows:

the first pair of the primers ID1: F: TGAGATGTGGCCATTAAGGA (SEQ ID NO:1);

R: TGGCAAAAGATCTTATATTTACTTCG (SEQ ID NO:2);

the second pair of the primers ID2: F: ACGGCTAAACGGTACTGCAT (SEQ ID NO:3);

R: ACACCAAGGGTGAAAAGTGG (SEQ ID NO:4);

the third pair of the primers ID3: F: ACCTCATCATGCTGAACGTG (SEQ ID NO:5);

R: TGAGGAACTCCGACTTCTGG (SEQ ID NO:6);

the fourth pair of the primers ID4: F: CAACTAAAACCAACACAAAATCCA (SEQ ID NO:7);

R: TGTCTAGTTGCATGTCTGAGTGTC (SEQ ID NO:8);

the fifth pair of the primers ID5: F: CTCTGAGGTAGCAGCCATCG (SEQ ID NO:9); R: TTAACCACACGCGGTTGC (SEQ ID NO:10);

the sixth pair of the primers ID6: F: GAGTTCGGCGACAGTCAGT (SEQ ID NO:11); R: TTGAAACATCCACGAATCTCA (SEQ ID NO:12);

the seventh pair of the primers ID7: F: GGCAAGATTGGATTGAGGAG (SEQ ID NO:13);

R: TCGCCAAACGAAAAGAAAAT (SEQ ID NO:14);

the eighth pair of the primers ID8: F: ATGGAACGCATGACATGAAA (SEQ ID NO:15);

R: CATCAAGAGGAGGGCAAAAA (SEQ ID NO:16); and

the ninth pair of the primers ID9: F: AATTCTTATGGACGGATACGC (SEQ ID NO:17);

R: TCAGCATCTCGTAAGCAAAAA (SEQ ID NO:18).

The applications of the primer groups described above include: the amplification of a backbone parent by using at least 4 pairs of primers in the primer groups described above; or the purity detection of hybrid rice by using at least one pair of primers.

The backbone parent materials of the hybrid rice are Quan9311A, Zhenshan 97A, 229A, Jufeng A, Y58S, C815S, Pei'ai 64S, Guangzhan 63S, E-nong 1S, Longke 638S, Jing 4155S, R534, Huahui 1308, R1377, Yuejingsimiao 2, Yuehesimiao, E'fengsimiao 1, Huazhan, Huanghuazhan, Fengxianghui 1, YR343, Xiang 5, 9311, R476, Feng 3592, Minghui 63, Shuhui 527, R1128 or R60.

The applications described above include the identification codes of the 29 backbone parents, and a process of using the primers includes: amplifying 29 backbone materials respectively by using 9 primers, wherein two band patterns are obtained for each primer with respect to the 29 backbone materials in the invention; and distinguishing and designating the two band patterns based on a size of a main band, thereby obtaining a 9-digit identification code corresponding to each variety,

Compared with the prior art, the invention has the following advantages:

1. Both time and labor are saved and the cost is reduced: the traditional variety identification is generally performed by planting the materials in the field and determining differences between varieties by investigating and recording a plurality phenotype, where a phenotype involves a plurality of characters from a seedling stage to a maturation stage, therefore, the whole growth period of rice needs to be observed to complete the identification; and by using the molecular identification number and the identification marker, DNA only needs to be extracted from a sample indoor and then tested by using the molecular marker, thereby saving time, labor and cost.

2. The results are more accurate and more reliable: as described above, the traditional variety identification depends on the differences in crop phenotypes, and the interaction between the genotype and the environment would lead to the change in gene expression to consequently lead to unstable phenotype, therefore, the records on field phenotypes are often affected by natural environment, field management, cultivation and other environmental factors to result in phenotype differences; however, by means of molecular marker, the detection can be implemented directly at a genotype level, and the results are thus more accurate and more reliable.

3. The primer groups according to the invention can distinguish any one of the 29 backbone parental materials simply by judging based on a band pattern, without sequencing; and meanwhile, the primers according to the invention have high polymorphism and the purity detection can be conducted by using only one pair of primers.

### BRIEF DESCRIPTION OF THE DRAWINGS

FIG. 1 illustrates a schematic diagram of band patterns of 29 hybrid rice backbone parents detected by using a primer ID1.

FIG. 2 illustrates a schematic diagram of the purity of a commercial hybrid rice seed Guangliangyou 476 detected by using a specific identification marker ID2 according to the invention;

Note: a lane M represents a marker; a lane 1 represents Guangzhan 63S which is a female parent of Guangliangyou 476; a lane 2 represents R476 which is a male parent of Guangliangyou 476; and lanes 3-76 represent DNA samples extracted from randomly selected germinated seeds, and arrows in the drawings indicate genotype seeds of mixed female parents.

### DETAILED EMBODIMENTS

Experimental methods that are not particularly explained in the embodiments are conventional molecular biological methods. Tag enzyme and dNTP used in this study are both manufactured by Shanghai Biocolor BioScience Co., Ltd. The rest are conventional biochemical reagents.

### Embodiment 1

Development and Screening of Primer Groups for Detecting Hybrid Rice Backbone Parent:

The applicant selected hybrid rice parent materials that had been widely used in the rice production at present, including a two-line sterile line, a three-line sterile line and a restorer line, and a total of 29 materials (Table 2) were used as study objects for developing their detecting primer groups. First, sequencing primers were designed randomly in the whole genome by using Nipponbare, a variety that had been sequenced; according to a pedigree relationship, two representative varieties having a distant genetic relationship, namely, a hybrid rice female parent Pei'ai 64S and a hybrid rice male parent Huazhan, with numerous combinations and wide promotion scope among the hybrid rice backbone parents were selected; the two varieties were subjected to sequence amplification and sequenced comparison by using the designed sequencing primers to obtain indel sites; and primers were designed by using the information of sequences on both sides of the sites to obtain ID markers.

The markers were selected based on the principle of uniform distribution in a rice genome. The applicant performed PCR amplification on 29 materials. PCR products were separated by using polyacrylamide gel electrophoresis to obtain electrophoretograms, based on which polymorphism index contents (PIC) were calculated for each pair of primers, with a computational formula as follows: PIC=1−Σ(Pi)², wherein Pi represents the gene frequency of indel primers at an i^{th} polymorphism site. PIC was an indicator of a degree of variation of the indel marker, showing the polymorphism of the indel DNA, wherein a high PIC indicates the indel marker has a strong capability of distinguishing different varieties. By selecting primers having high PIC, the varieties under test could be distinguished by using minimum marker combinations. 9 pairs of primers were finally screened to form primer combinations capable of distinguishing 29 parent materials. The primers were as follows:

TABLE 1
Primers adapted to detecting 29 hybrid
rice backbone parent materials

| | | | PCR | | |
|---|---|---|---|---|---|
| | Identi- | | prod- | | |
| | fica- | Marker | uct | Annealing | |
| | tion | sequence | Size | temper- | |
| No. | Name | (5′→3′) | (bp) | ature | PIC |
| 1 | ID1 | F: TGAGATGT | 163 | 59 | 0.60 |
| | | GGCCATTAAGG | | | |
| | | A | | | |
| | | (SEQ ID | | | |
| | | NO: 1) | | | |
| | | R: TGGCAAAAG | | | |
| | | ATCTTATATTTA | | | |
| | | CTTCG | | | |
| | | (SEQ ID | | | |
| | | NO: 2) | | | |
| 2 | ID2 | F: ACGGCTAAA | 226 | 60 | 0.56 |
| | | CGGTACTGCAT | | | |
| | | (SEQ ID | | | |
| | | NO: 3) | | | |
| | | R: ACACCAAGG | | | |
| | | GTGAAAAGTGG | | | |
| | | (SEQ ID | | | |
| | | NO: 4) | | | |
| 3 | ID3 | F: ACCTCATCA | 219 | 60 | 0.54 |
| | | TGCTGAACGTG | | | |
| | | (SEQ ID | | | |
| | | NO: 5) | | | |
| | | R: TGAGGAACT | | | |
| | | CCGACTTCTGG | | | |
| | | (SEQ ID | | | |
| | | NO: 6) | | | |
| 4 | ID4 | F: CAACTAAAA | 130 | 60 | 0.52 |
| | | CCAACACAAAAT | | | |
| | | CCA | | | |
| | | (SEQ ID | | | |
| | | NO: 7) | | | |
| | | R: TGTCTAGTT | | | |
| | | GCATGTCTGAGT | | | |
| | | GTC | | | |
| | | (SEQ ID | | | |
| | | NO: 8) | | | |
| 5 | ID5 | F: CTCTGAGGT | 100 | 60 | 0.51 |
| | | AGCAGCCATCG | | | |
| | | (SEQ ID | | | |
| | | NO: 9) | | | |
| | | R: TTAACCACA | | | |
| | | CGCGGTTGC | | | |
| | | (SEQ ID | | | |
| | | NO: 10) | | | |
| 6 | ID6 | F: GAGTTCGGC | 176 | 59 | 0.51 |
| | | GACAGTCAGT | | | |
| | | (SEQ ID | | | |
| | | NO: 11) | | | |
| | | R: TTGAAACAT | | | |
| | | CCACGAATCTCA | | | |
| | | (SEQ ID | | | |
| | | NO: 12) | | | |
| 7 | ID7 | F: GGCAAGATT | 151 | 60 | 0.49 |
| | | GGATTGAGGAG | | | |
| | | (SEQ ID | | | |
| | | NO: 13) | | | |
| | | R: TCGCCAAAC | | | |
| | | GAAAAGAAAAT | | | |
| | | (SEQ ID | | | |
| | | NO: 14) | | | |
| 8 | ID8 | F: ATGGAACGC | 159 | 60 | 0.47 |
| | | ATGACATGAAA | | | |
| | | (SEQ ID | | | |
| | | NO: 15) | | | |
| | | R: CATCAAGAG | | | |
| | | GAGGGCAAAAA | | | |
| | | (SEQ ID | | | |
| | | NO: 16) | | | |
| 9 | ID9 | F: AATTCTTAT | 159 | 58 | 0.47 |
| | | GGACGGATACGC | | | |
| | | (SEQ ID | | | |
| | | NO: 17) | | | |
| | | R: TCAGCATCT | | | |
| | | CGTAAGCAAAAA | | | |
| | | (SEQ ID | | | |
| | | NO: 18) | | | |

Note:
F represents a forward primer; R represents a reverse primer; PIC: polymorphism index contents; and the primer product mentioned above refers to a fragment size of each primer in a reference genome Nipponbare.

### Embodiment 2

Application of Primer Groups to Detection of Hybrid Rice Backbone Parent

In this embodiment, the applicant gave names based on the band sizes of the primers with respect to the 29 parent materials, whereby the codes, hereinafter referred to identification codes, were composed. A method was as follows:

29 parent materials as shown in FIG. 2 were amplified respectively by using the 9 pairs of primers according to the invention; a total of two bands different in size were obtained for each primer among the 29 materials; and a person skilled in the art may give names based on band position (or band size).

An amplification method specifically included:

1) DNAs of the materials shown in Table 2 were extracted b using a CTAB method, wherein the primers were those corresponding to the polymorphism markers ID1-ID9 developed in Embodiment 1.

2) PCR

The PCR reaction system was 20 μL, containing 2.0 μL of 10×Buffer, 1.2 μL of dNTPs (10 mmol/L), two primers (0.2 μM for each), 0.2 μL of Taq enzyme (5 U/μL), 2.0 μL of template DNA, and 12.8 μL of ddH2O. PCR procedures were as follows: predegeneratiion was conducted at 94° C. for 5 min; degeneration was conducted at 94° C. for 1 min, annealing was conducted at 60° C. for 45 s, and extending was conducted at 72° C. for 1 min, with 32 cycles in total; then, extending was conducted at 72° C. for 10 min, placing an amplification product in 6% PAGE gel for electrophoresis, and silver staining and developing were subsequently conducted and then results were recorded.

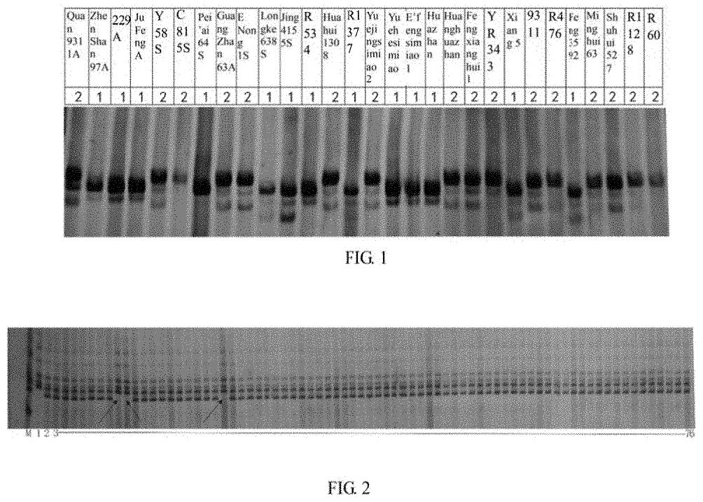

The naming rules in the invention were illustrated by taking ID1 as an example, where 29 parent materials were amplified by using ID1, with amplification results shown in FIG. 1; a total of two band patterns were obtained for ID1 among the 29 materials; due to band mixing occurring during the amplification in the invention, the distinguishing was conducted mainly based on the size of a main band in the invention; the applicant named a small band as “1” and a big band as “2”; and consequently, among the identification codes in the invention, Quan 9311A had a first digit as 2, and Zhenshan 97A had a first digit as 1. By using ID2, two bands different in size were also obtained by amplifying the 29 parental materials, the small band was named as “1”, the big band was named as “2”, and so on in a similar fashion, the electrophoretograms and corresponding names were obtained by amplifying the 29 parent materials using the 9 primers. The details were shown as in Table 2:

TABLE 2

| No. | Parent | ID1 | ID2 | ID3 | ID4 | ID5 | ID6 | ID7 | ID8 | ID9 |
|---|---|---|---|---|---|---|---|---|---|---|
| 1 | Quan 9311A | 2 | 1 | 1 | 2 | 1 | 1 | 1 | 1 | 1 |
| 2 | Zhengshan 97A | 1 | 1 | 1 | 1 | 2 | 1 | 1 | 1 | 2 |
| 3 | 229A | 1 | 2 | 1 | 1 | 1 | 2 | 1 | 1 | 1 |
| 4 | Jufeng A | 1 | 2 | 1 | 1 | 1 | 2 | 1 | 1 | 2 |
| 5 | Y58S | 2 | 2 | 2 | 1 | 2 | 1 | 2 | 1 | 1 |
| 6 | C815S | 2 | 2 | 2 | 1 | 2 | 1 | 2 | 2 | 2 |
| 7 | Pei'ai 64S | 1 | 2 | 2 | 1 | 2 | 2 | 2 | 2 | 2 |
| 8 | Guangzhan 63S | 2 | 2 | 2 | 1 | 2 | 2 | 1 | 1 | 2 |
| 9 | E-nong 1S | 2 | 1 | 1 | 1 | 2 | 2 | 1 | 1 | 2 |
| 10 | Longke 638S | 1 | 2 | 1 | 1 | 1 | 1 | 2 | 1 | 2 |
| 11 | Jing 4155S | 1 | 1 | 1 | 1 | 1 | 2 | 1 | 2 | 2 |
| 12 | R534 | 1 | 1 | 2 | 1 | 1 | 2 | 1 | 2 | 2 |
| 13 | Huahui 1308 | 2 | 2 | 2 | 2 | 2 | 1 | 2 | 2 | 2 |
| 14 | R1377 | 1 | 2 | 2 | 1 | 1 | 2 | 2 | 1 | 2 |
| 15 | Yuejingsimiao 2 | 2 | 2 | 2 | 2 | 1 | 1 | 2 | 1 | 2 |
| 16 | Yuehesimiao | 1 | 1 | 2 | 2 | 2 | 1 | 2 | 1 | 2 |
| 17 | E'fengsimiao 1 | 1 | 1 | 1 | 2 | 2 | 1 | 1 | 1 | 2 |
| 18 | Huazhan | 1 | 1 | 1 | 2 | 2 | 2 | 1 | 1 | 2 |
| 19 | Huanghuazhan | 2 | 1 | 1 | 2 | 1 | 1 | 1 | 2 | 2 |
| 20 | Fengxianghui 1 | 2 | 2 | 2 | 1 | 1 | 1 | 1 | 1 | 1 |
| 21 | YR343 | 2 | 1 | 2 | 2 | 1 | 1 | 1 | 1 | 1 |
| 22 | Xiang 5 | 1 | 2 | 2 | 2 | 1 | 1 | 1 | 1 | 1 |
| 23 | 9311 | 2 | 2 | 1 | 2 | 1 | 1 | 1 | 1 | 1 |
| 24 | R476 | 2 | 1 | 1 | 2 | 1 | 2 | 1 | 1 | 1 |
| 25 | Feng 3592 | 1 | 1 | 1 | 1 | 2 | 2 | 2 | 2 | 1 |
| 26 | Minghui 63 | 2 | 1 | 1 | 1 | 1 | 2 | 2 | 2 | 1 |
| 27 | Shuhui 527 | 2 | 2 | 1 | 1 | 2 | 1 | 2 | 1 | 1 |
| 28 | R1128 | 2 | 2 | 2 | 1 | 1 | 2 | 2 | 2 | 1 |
| 29 | R60 | 2 | 1 | 2 | 1 | 2 | 2 | 2 | 2 | 2 |

In fact, since the primers screened according to the invention had good polymorphism, it was unnecessary to simultaneously use the 9 pairs of primers when each material was identified. Based on Table 2, the applicant further screened a combination of minimum specific identification primers for each material. As shown in Table 3, in case of the first variety Quan 9311A, a total of 8 pair of primers, namely, ID1-ID8, were required to distinguish it from all the other varieties; and if it needed to be distinguished from one or several varieties, the markers with different numbers between the varieties were only required to be selected.

Table 3 Combination of minimum specific identification primers as determined for each material based on band patterns of electrophoretograms of 9 pairs of primers

| | | | | | | | | | | | Combination of |
|---|---|---|---|---|---|---|---|---|---|---|---|
| | | | | | | | | | | | identification |
| No. | Parent | ID1 | ID2 | ID3 | ID4 | ID5 | ID6 | ID7 | ID8 | ID9 | primers |
| 1 | Quan 9311A | 2 | 1 | 1 | 2 | 1 | 1 | 1 | 1 | | ID1-ID8 |
| 2 | Zhengshan | 1 | 1 | 1 | 1 | 2 | 1 | | | | ID1-ID6 |
| 3 | 229A | 1 | 2 | 1 | 1 | 1 | 2 | 1 | 1 | 1 | ID1-ID9 |
| 4 | Jufeng A | 1 | 2 | 1 | 1 | 1 | 2 | 1 | 1 | 2 | ID1-ID9 |
| 5 | Y58S | 2 | 2 | 2 | 1 | 2 | 1 | 2 | 1 | | ID1-ID8 |
| 6 | C815S | 2 | 2 | 2 | 1 | 2 | 1 | 2 | 2 | | ID1-ID8 |
| 7 | Pei'ai 64S | 1 | 2 | 2 | 1 | 2 | | | | | ID1-ID5 |
| 8 | Guangzhan | 2 | 2 | 2 | 1 | 2 | 2 | | | | ID1-ID6 |
| 9 | E-nong 1S | 2 | 1 | 1 | 1 | 2 | | | | | ID1-ID5 |
| 10 | Longke 638S | 1 | 2 | 1 | 1 | 1 | 1 | | | | ID1-ID6 |
| 11 | Jing 4155S | 1 | 1 | 1 | 1 | 1 | | | | | ID1-ID5 |
| 12 | R534 | 1 | 1 | 2 | 1 | | | | | | ID1-ID4 |
| 13 | Huahui 1308 | 2 | 2 | 2 | 2 | 2 | | | | | ID1-ID5 |
| 14 | R1377 | 1 | 2 | 2 | 1 | 1 | | | | | ID1-ID5 |
| 15 | Yuejingsimiao | 2 | 2 | 2 | 2 | 1 | | | | | ID1-ID5 |
| 16 | Yuehesimiao | 1 | 1 | 2 | 2 | | | | | | ID1-ID4 |
| 17 | E'fengsimiao 1 | 1 | 1 | 1 | 2 | 2 | 1 | | | | ID1-ID6 |
| 18 | Huazhan | 1 | 1 | 1 | 2 | 2 | 2 | | | | ID1-ID6 |
| 19 | Huanghuazhan | 2 | 1 | 1 | 2 | 1 | 1 | 1 | 2 | | ID1-ID8 |
| 20 | Fengxianghui 1 | 2 | 2 | 2 | 1 | 1 | 1 | | | | ID1-ID6 |
| 21 | YR343 | 2 | 1 | 2 | 2 | | | | | | ID1-ID4 |
| 22 | Xiang 5 | 1 | 2 | 2 | 2 | | | | | | ID1-ID4 |
| 23 | 9311 | 2 | 2 | 1 | 2 | | | | | | ID1-ID8 |
| 24 | R476 | 2 | 1 | 1 | 2 | 1 | 2 | | | | ID1-ID6 |
| 25 | Feng 3592 | 1 | 1 | 1 | 1 | 2 | 2 | | | | ID1-ID6 |
| 26 | Minghui 63 | 2 | 1 | 1 | 1 | 1 | | | | | ID1-ID5 |
| 27 | Shuhui 527 | 2 | 2 | 1 | 1 | | | | | | ID1-ID4 |
| 28 | R1128 | 2 | 2 | 2 | 1 | 1 | 2 | | | | ID1-ID6 |
| 29 | R60 | 2 | 1 | 2 | 1 | | | | | | ID1-ID4 |

From Table 2 and Table 3, the primer groups according to the invention can completely distinguish 29 parental materials.

### Example 3

Application of primer groups to detection of hybrid rice backbone parent (purity detection):

Based on the identification codes of the hybrid rice parent in Embodiment 2, the identification code of Guangzhan 3S had a corresponding value of 2 under ID2; the identification code of R476 had a corresponding value of 1 under ID2. Consequently, ID2 shown polymorphism between the two parents and could distinguish them, and thus, ID2 was selected to detect the purity of hybrid seeds.

1) Biological Material

The lane M represented a marker; the lane 1 represented Guangzhan 63S which was the female parent of Guangliangyou 476; the lane 2 represented R476 which was the male parent of Guangliangyou 476; and the remaining lanes 3-76 represented seeds that were randomly selected from hybrid seeds Guangliangyou 476.

2) DNA Extraction of Rice and Primers

DNA of the above-mentioned material was extracted using a CTAB method.

3) PCR

The PCR reaction system was 20 μL, containing 2.0 μL of 10×Buffer, 1.2 μL of dNTPs (10 mmol/L), two primers (0.2 μM for each), 0.2 μL of Taq enzyme (5 U/μL), 2.0 μL of template DNA, and 12.8 μL of ddH2O. PCR procedures were as follows: predegeneratiion was conducted at 94° C. for 5 min; degeneration was conducted at 94° C. for 1 min, annealing was conducted at 60° C. for 45 s, and extending was conducted at 72° C. for 1 min, with 32 cycles in total; then, extending was conducted at 72° C. for 10 min, placing an amplification product in 6% PAGE gel for electrophoresis, and silver staining and developing were subsequently conducted and then results were recorded.

4). Analysis of Results

The lane 1 represented Guangzhan 63S which was the female parent of Guangliangyou 476; the lane 2 represented R476 which was the male parent of Guangliangyou 476; and the remaining lanes 3-76 represented DNA samples extracted from randomly selected germinated seeds of the hybrid seeds Guangliangyou 476. As shown in the electrophoretograms in FIG. 2, a total of 74 individual samples were detected, where 3 band patterns were shown as band patterns of homozygous female parents, and the rest were heterozygous band patterns from both parents. Therefore, it was predicted that this batch of seeds had the purity of about 95.9%. Meanwhile, we planted 500 seeds from this batch in the field, and conducted a growth-out test to identify that hybrid plants were all female parents with the purity of 96.1%, which was basically consistent with the results from marker detection.

Therefore, the application process in this embodiment can be effectively applied to the purity detection of hybrid rice seeds.

1: A primer group for detecting a hybrid rice backbone parent, comprising:
a first group of primers ID1 being F: TGAGATGTGGCCATTAAGGA (SEQ ID NO:1) and R: TGGCAAAAGATCTTATATTTACTTCG (SEQ ID NO:2);
a second group of primers ID2 being F: ACGGCTAAACGGTACTGCAT (SEQ ID NO:3) and R: ACACCAAGGGTGAAAAGTGG (SEQ ID NO:4);
a third group of primers ID3 being F: ACCTCATCATGCTGAACGTG (SEQ ID NO:5) and R: TGAGGAACTCCGACTTCTGG (SEQ ID NO:6);
a fourth group of primers ID4 being F: CAACTAAAACCAACACAAAATCCA (SEQ ID NO:7) and R: TGTCTAGTTGCATGTCTGAGTGTC (SEQ ID NO:8);
a fifth group of primers ID5 being F: CTCTGAGGTAGCAGCCATCG (SEQ ID NO:9) and R: TTAACCACACGCGGTTGC (SEQ ID NO:10);
a sixth group of primers ID6 being F: GAGTTCGGCGACAGTCAGT (SEQ ID NO:11) and R: TTGAAACATCCACGAATCTCA (SEQ ID NO:12);
a seventh group of primers ID7 being F: GGCAAGATTGGATTGAGGAG (SEQ ID NO:13) and R: TCGCCAAACGAAAAGAAAAT (SEQ ID NO:14);
an eighth group of primers ID8 being F: ATGGAACGCATGACATGAAA (SEQ ID NO:15) and R: CATCAAGAGGAGGGCAAAAA (SEQ ID NO:16);
a ninth group of primers ID9 being F: AATTCTTATGGACGGATACGC (SEQ ID NO:17) and R: TCAGCATCTCGTAAGCAAAAA (SEQ ID NO:18).

2-4. (canceled)

5: A method for detecting a hybrid rice backbone parent according to claim 1, comprising
applying the primer group as described in claim 1 for detecting a hybrid rice backbone parent,
wherein the hybrid rice backbone parent is Quan9311A, Zhenshan 97A, 229A, Jufeng A, Y58S, C815S, Pei'ai 64S, Guangzhan 63S, E-nong 1S, Longke 638S, Jing 4155S, R534, Huahui 1308, R1377, Yuejingsimiao 2, Yuehesimiao, E'fengsimiao 1, Huazhan, Huanghuazhan, Fengxianghui 1, YR343, Xiang 5, 9311, R476, Feng 3592, Minghui 63, Shuhui 527, R1128, or R60.

6: A method for detecting purity of a hybrid rice seed according to claim 1, comprising
applying the primer group as described in claim 1 in purity detection of a hybrid rice seed,
wherein the hybrid rice seed is Quan9311A, Zhenshan 97A, 229A, Jufeng A, Y58S, C815S, Pei'ai 64S, Guangzhan 63S, E-nong 1S, Longke 638S, Jing 4155S, R534, Huahui 1308, R1377, Yuejingsimiao 2, Yuehesimiao, E'fengsimiao 1, Huazhan, Huanghuazhan, Fengxianghui 1, YR343, Xiang 5, 9311, R476, Feng 3592, Minghui 63, Shuhui 527, R1128, or R60.

7: A method for establishing a hybrid rice identification code according to claim 1, comprising
applying the primer group as described in claim 1 to establish a hybrid rice identification code by amplifying 29 backbone materials respectively by using 9 primers to obtain two band patterns for each primer with respect to the 29 backbone materials, and
distinguishing and naming the two band patterns based on a size of a main band, thereby obtaining a 9-digit identification code corresponding to each variety,
wherein the hybrid rice backbone parent is Quan9311A, Zhenshan 97A, 229A, Jufeng A, Y58S, C815S, Pei'ai 64S, Guangzhan 63S, E-nong 1S, Longke 638S, Jing 4155S, R534, Huahui 1308, R1377, Yuejingsimiao 2, Yuehesimiao, E'fengsimiao 1, Huazhan, Huanghuazhan, Fengxianghui 1, YR343, Xiang 5, 9311, R476, Feng 3592, Minghui 63, Shuhui 527, R1128, or R60.


SEQUENCE LISTING (18 sequences)

SEQ ID NO: 1 - DNA, 20 bp, Artificial Sequence
  Primer ID1 Front
tgagatgtggccattaagga

SEQ ID NO: 2 - DNA, 26 bp, Artificial Sequence
  Primer ID1 Rear
tggcaaaagatcttatatttacttcg

SEQ ID NO: 3 - DNA, 20 bp, Artificial Sequence
  Primer ID2 Front
acggctaaacggtactgcat

SEQ ID NO: 4 - DNA, 20 bp, Artificial Sequence
  Primer ID2 Rear
acaccaagggtgaaaagtgg

SEQ ID NO: 5 - DNA, 20 bp, Artificial Sequence
  Primer ID3 Front
acctcatcatgctgaacgtg

SEQ ID NO: 6 - DNA, 20 bp, Artificial Sequence
  Primer ID3 Rear
tgaggaactccgacttctgg

SEQ ID NO: 7 - DNA, 24 bp, Artificial Sequence
  Primer ID4 Front
caactaaaaccaacacaaaatcca

SEQ ID NO: 8 - DNA, 24 bp, Artificial Sequence
  Primer ID4 Rear
tgtctagttgcatgtctgagtgtc

SEQ ID NO: 9 - DNA, 20 bp, Artificial Sequence
  Primer ID5 Front
ctctgaggtagcagccatcg

SEQ ID NO: 10 - DNA, 18 bp, Artificial Sequence
  Primer ID5 Rear
ttaaccacacgcggttgc

SEQ ID NO: 11 - DNA, 19 bp, Artificial Sequence
  Primer ID6 Front
gagttcggcgacagtcagt

SEQ ID NO: 12 - DNA, 21 bp, Artificial Sequence
  Primer ID6 Rear
ttgaaacatccacgaatctca

SEQ ID NO: 13 - DNA, 20 bp, Artificial Sequence
  Primer ID7 Front
ggcaagattggattgaggag

SEQ ID NO: 14 - DNA, 20 bp, Artificial Sequence
  Primer ID7 Rear
tcgccaaacgaaaagaaaat

SEQ ID NO: 15 - DNA, 20 bp, Artificial Sequence
  Primer ID8 Front
atggaacgcatgacatgaaa

SEQ ID NO: 16 - DNA, 20 bp, Artificial Sequence
  Primer ID8 Rear
catcaagaggagggcaaaaa

SEQ ID NO: 17 - DNA, 21 bp, Artificial Sequence
  Primer ID9 Front
aattcttatggacggatacgc

SEQ ID NO: 18 - DNA, 21 bp, Artificial Sequence
  Primer ID9 Rear
tcagcatctcgtaagcaaaaa